(19) United States
(12) Patent Application Publication
(10) Pub. No.: US 2021/0321638 A1
(43) Pub. Date: Oct. 21, 2021

(54) ANIMAL FEED AND METHODS TO PROVIDE SUCH FEED

(71) Applicant: Nutreco Nederland B.V., Boxmeer (NL)

(72) Inventors: Petra Johanna Roubos-van den Hil, Boxmeer (NL); Mark Bouwens, Boxmeer (NL)

(21) Appl. No.: 16/326,444

(22) PCT Filed: Aug. 25, 2017
(86) PCT No.: PCT/NL2017/050560

§ 371 Date: Feb. 19, 2019

(30) Foreign Application Priority Data
Aug. 26, 2016 (NL) 2017376

(51) Int. Cl. A23K 10/30; A23K 20/105; A23K 20/163; A23K 50/30; A23K 50/75; A61K 31/7016; A61K 36/07

(52) U.S. Cl. CPC A23K 10/30; A23K 20/105; A23K 20/163; A61K 36/07; A23K 50/75; A61K 31/7016; A23K 50/30

(57) ABSTRACT

The disclosure pertains to a composition comprising mycelium of Agaricus Blazei Murill and an agent-affecting bacteria belonging to Enterobacteriaceae. The composition has a positive effect on the shedding of bacteria of the Enterobacteriaceae, in particular, of Salmonella and/or Escherichia. Moreover, the composition is effective against resistant bacteria of the Enterobacteriaceae.

### CROSS-REFERENCE TO RELATED APPLICATIONS

This application is a national phase entry under 35 U.S.C. § 371 of International Patent Application PCT/NL2017/050560, filed Aug. 25, 2017, designating the United States of America and published in English as International Patent Publication WO 2018/038616 A1 on Mar. 1, 2018, which claims the benefit under Article 8 of the Patent Cooperation Treaty to The Netherlands Patent Application Serial No. 2017376, filed Aug. 26, 2016.

### TECHNICAL FIELD

This disclosure pertains to animal feed in general.

### BACKGROUND

Animal feed is used to support the normal metabolism of animals in order for these animals to stay healthy and grow to their full capabilities. In particular, for food-producing animals, it is very important that the feed optimally supports their health since this is often reflected in their performance as measured by establishing the average daily weight gain of an animal, its weight at the age of slaughter or its age at the slaughter weight. In particular, in raising food-producing animals, much attention is given to control the spreading of bacteria within a group of animals kept at a particular production site. In particular, since such bacteria might be pathogenic to the animal itself (infection thus reducing the animal's health status and hence its performance) or possibly also to consumers of the animal (humans or other animals). Common methods to reduce spreading of bacteria are to use antibiotics, and/or to vaccinate the animal. Another method used is containment (quarantine) of the animal in combination with sterilizing its feed. This method, however, is not suitable to raise animals for consumption purposes because of the high costs involved.

### BRIEF SUMMARY

An animal feed that is particularly suitable to improve animal wellbeing, preferably to reduce or mitigate infection with ubiquitous pathogenic bacteria such as bacteria belonging to the Enterobacteriaceae, in particular, Salmonella species and Escherichia species, is provided.

A composition comprising mycelium of Agaricus Blazei Murill and an agent-affecting bacteria belonging to Enterobacteriaceae, in particular, an agent-affecting bacteria belonging to Salmonella and/or Escherichia, is disclosed. The composition of the disclosure generally has a positive effect on the shedding of bacteria of the Enterobacteriaceae, in particular, of Salmonella and/or Escherichia. Moreover, the composition is effective against resistant bacteria of the Enterobacteriaceae, in particular, of Salmonella and/or Escherichia. The composition may further reduce the bacteria infestation in the infested animal leading to an animal with an improved health status and an increased growth performance. In particular, it was found that the colonization with pathogenic bacteria belonging to the group of the enterobacteriacea is reduced, which is demonstrated by a lower bacteria count in the animal's feces. With the composition disclosed herein, it may be possible to reduce the amount of preventative antibiotics administered to animals.

The composition of the disclosure includes animal feed, a premix of animal feed and a feed additive. Consequently, the disclosure further comprises to a feed additive comprising mycelium of Agaricus Blazei Murill and an agent-affecting bacteria belonging to Enterobacteriaceae, in particular, an agent-affecting bacteria belonging to Salmonella and/or Escherichia. Such a feed additive may comprise further ingredients commonly used in feed additives. The feed additive of the disclosure may be applied and/or added to a premix of animal feed, to animal feed and/or to drinking water. It may be applied to preserve the premix and/or the feed. The feed additive may further be used to improve the gut health of the animal.

The disclosure further comprises a premix of animal feed comprising mycelium of Agaricus Blazei Murill and an agent-affecting bacteria belonging to Enterobacteriaceae, in particular, an agent-affecting bacteria belonging to Salmonella and/or Escherichia. The premix of the disclosure may comprise further ingredients commonly used in premixes of animal feed. The premixes of the disclosure generally are further processed and further ingredients are added to form animal feed. Hence, the disclosure also comprises an animal feed comprising mycelium of Agaricus Blazei Murill and an agent-affecting bacteria belonging to Enterobacteriaceae, in particular, an agent-affecting bacteria belonging to Salmonella and/or Escherichia. The animal feed is generally fed to the animals. Animal feed generally comprises animal nutrients such as fats and/or proteins and/or carbohydrates that is fed to an animal to provide in its metabolic requirements. Animal feed can be a nutritionally complete feed (i.e., providing all required nutrients to support a normal metabolism of the animal). Similar ingredients are also contained in a premix of animal feed that, however, contains only part of the required nutrients, and needs to be mixed with other nutrients or fed separately from these other nutrients.

It is noted that Agaricus Blazei Murill is also called Agaricus Blazei Brasiliensis, Agaricus subrufescens, or Agaricus rufotegulis. In this application, the common name Agaricus Blazei Murill will be used. Hereinbelow, the abbreviation ABM and the term Agaricus Blazei Murill are used interchangeably.

The mycelium of ABM used in the compositions of the disclosure can be mycelium per se as grown in a liquid matrix or can be mycelium grown on grain. In the present disclosure, mycelium of ABM grown on grain is preferred. The grain used for the fermentation can be any grain known in the art suitable for this purpose. Examples of such grains include corn, wheat, millet, sorghum, barley, rye, oat, and soy beans. Preferred grains are millet, oat and rye, and most preferred is rye. More details on mycelium of ABM grown on grain including the preparation techniques are described in WO 2013/171194, which is incorporated herein by reference.

The amount of mycelium of ABM grown on the grain can be chosen as desired where the level of fermentation will determine the amount. Typically, the amount of mycelium is at least 1 weight percent (wt %) and at most 50 wt %, based on the total weight of mycelium and grain. Preferably, the amount of mycelium is at least 5 wt % and most preferably at most 10 wt %, and preferably at most 40 wt %, more preferably at most 35 wt % and most preferably at most 30 wt %, based on total weight of mycelium and grain. Methods to determine the amount of mycelium, such as measuring the ergosterol content, are described in WO 2013/171194.

In one aspect, the amount of mycelium of ABM is at least 0.001 wt %, preferably at least 0.01 wt %, more preferably at least 0.1 wt % and most preferably at least 0.5 wt %, and at most 30 wt %, preferably at most 20 wt %, more preferably at most 15 wt %, and most preferably at most 10 wt %, based on the total weight of the composition.

In another aspect, the amount of mycelium of ABM is at least 0.1 wt %, preferably at least 0.5 wt %, more preferably at least 1 wt % and most preferably at least 2 wt %, and at most 30 wt %, preferably at most 20 wt %, more preferably at most 15 wt %, and most preferably at most 10 wt %, based on the total weight of the feed additive.

In yet another aspect, the amount of mycelium of ABM is at least 0.001 wt %, preferably at least 0.01 wt %, more preferably at least 0.1 wt % and most preferably at least 0.15 wt %, and at most 5 wt %, preferably at most 2 wt %, more preferably at most 1 wt %, and most preferably at most 0.5 wt %, based on the total weight of the animal feed.

In yet a further aspect, the amount of mycelium of ABM is at least 0.01 wt %, preferably at least 0.1 wt %, more preferably at least 1 wt % and most preferably at least 1.5 wt %, and at most 15 wt %, preferably at most 12 wt %, more preferably at most 10 wt %, and most preferably at most 5 wt %, based on the total weight of the premix of animal feed.

The agent-affecting bacteria belonging to enterobacteriaceae of the disclosure can be any such agent known in the art. Such agents include pharmaceutically active ingredients and nutritionally active ingredients. Pharmaceutically active ingredients are agents that are capable of killing the bacteria, and include antibiotics such as cephalosporins and fluoroquinolones. The combination with the mycelium of the disclosure may allow for a reduction in the amount of pharmaceutically active ingredient administered to the animal preventatively or curatively. Nutritionally active ingredients are agents that affect the gut health and/or the immune system of the animal and/or are capable of reducing the growth of the bacteria either in the animal feed and/or in the animal's gut. Examples of such agents include organic acids such as formic acid, lactic acid, butyrate and benzoic acid; prebiotic carbohydrates such as mono-, di- and polysaccharides of mannans, mono-, di- and polysaccharides of arabinoxylans, mono-, di- and polysaccharides of inulin-type fructans, galacto-oligosaccharides, xylo-oligosaccharides and chyto-oligosaccharides; prebiotic fibers such as pea fiber, palm kernel fiber, coconut fiber, wheat fiber, oat fiber and further processed products; plant extracts such as essential oils, carvacrol, thymol, eugenol, cinnemaldehyde and capsacin; and probiotics or microbial-derived products such as Bacillus spp., Saccharomyces spp., Lactobacillus spp. and fermented products or co-products thereof such as yeast cell-wall derived products Preferred agents are selected from the group consisting of formic acid, lactic acid, benzoic acid, hydrolyzed copra meal, yeast cell wall derived products, manno-oligosaccharides and β-1,4-mannobiose. Even more preferred is hydrolyzed copra meal, yeast cell wall derived products, manno-oligosaccharides and β-1,4-mannobiose. Even more preferred are manno-oligosaccharides and β-1,4-mannobiose. Most preferred is β-1,4-mannobiose.

In one aspect, the amount of agent-affecting bacteria belonging to enterobacteriaceae is at least 0.001 wt %, preferably at least 0.01 wt %, more preferably at least 0.1 wt % and most preferably at least 0.5 wt %, and at most 30 wt %, preferably at most 20 wt %, more preferably at most 15 wt %, and most preferably at most 10 wt %, based on the total weight of the composition.

In another aspect, the amount of agent-affecting bacteria belonging to enterobacteriaceae is at least 0.1 wt %, preferably at least 0.5 wt %, more preferably at least 1 wt % and most preferably at least 2 wt %, and at most 30 wt %, preferably at most 20 wt %, more preferably at most 15 wt %, and most preferably at most 10 wt %, based on the total weight of the feed additive.

In yet another aspect, the amount of agent-affecting bacteria belonging to enterobacteriaceae is at least 0.001 wt %, preferably at least 0.01 wt %, more preferably at least 0.1 wt % and most preferably at least 0.15 wt %, and at most 5 wt %, preferably at most 2 wt %, more preferably at most 1 wt %, and most preferably at most 0.5 wt %, based on the total weight of the animal feed.

In yet a further aspect, the amount of agent-affecting bacteria belonging to enterobacteriaceae is at least 0.01 wt %, preferably at least 0.1 wt %, more preferably at least 1 wt % and most preferably at least 1.5 wt %, and at most 15 wt %, preferably at most 12 wt %, more preferably at most 10 wt %, and most preferably at most 5 wt %, based on the total weight of the premix of animal feed.

In one embodiment of the disclosure, the weight ratio of mycelium of ABM and the agent-affecting bacteria belonging to enterobacteriaceae in the compositions of the disclosure is at least 0.01, preferably at least 0.1, more preferably at least 0.3 and most preferably at least 0.5, and at most 50, preferably at most 20, more preferably at most 10 and most preferably at most 5.

The compositions of the disclosure are effective against bacteria belonging to enterobacteriaceae. In particular, the compositions of the disclosure are effective against enterobacteriaceae that are pathogens for animals, in particular, for livestock such as chickens, pigs and ruminants. Such bacteria include Salmonella such as S. typhimurium, S. infantis, S. heidelberg, S. enteritidis, S. tennessee, S. brandenburg, S. newport, S. mbandaka, S. cubana, S. senftenberg, S. yoruba, S. derby, S. kedoudgo, S. cholerae-suis, S. livingstone, S. lexington, S. gallinarum and S. agona, and Escherichia such as E. coli and E. vulnerus. Preferred are agents affecting one or more bacteria selected from the group of S. typhimurium, S. enteritidis and E. coli.

It is noted that the mycelium of ABM and the agent-affecting bacteria belonging to enterobacteriaceae do not need to be present in the same feed material at the same time. It is essential that the various feed components (solid feed, drinking water etc.) taken by an animal as a whole comprise both ingredients, such that at least in the gastro-intestinal tract both ingredients are combined and act in accordance with this disclosure.

The disclosure also pertains to a kit-of-parts comprising a first constituting part that comprises Agaricus Blazei Murill (ABM) mycelium and a second constituting part comprising one or more agent-affecting bacteria belonging to enterobacteriaceae, and optionally an instruction to orally administer both these parts of the kit to an animal. It is noted that for the sense of the disclosure, the parts do not need to be present in one single container. It is foreseen that the parts are provided in separate containers, not packed together, but with the clear intention (for example, by indications provided on a web-site, separate leaflet, etc.) to be used according to the teaching of the disclosure, for example, by adding one or both parts to animal feed, and/or one or both parts to the drinking water that is offered to the animal in conjunction with its feed.

The disclosure also enables a method of feeding an animal by providing feed to the animal comprising Agaricus Blazei Murill mycelium and one or more agent-affecting bacteria belonging to enterobacteriaceae. This can be accomplished, for example, by having both ingredients present in the animal feed, or by feeding the animal with a first substance comprising the mycelium of ABM and a second substance comprising the agent-affecting bacteria belonging to enterobacteriaceae (for example, drinking water in which the acids are present). The disclosure also enables a method to provide animal feed by mixing Agaricus Blazei Murill mycelium and one or more agent-affecting bacteria belonging to enterobacteriaceae with protein and/or carbohydrates and/or fats to provide the feed.

In a first embodiment of the feed according to the disclosure, the ABM mycelium is present in the animal feed in an amount of at least 0.01 kg per ton of total daily intake, preferably at least 0.02, more preferably at least 0.05 kg per ton, and at most 10 kg per ton of total daily intake of feed, preferably at most 5 kg per ton, more preferably at most 2 kg per ton, and most preferably at most 1 kg per ton of total daily intake. In the context of this application, the total daily intake of feed is the complete mass of feed an animal takes per day, excluding drinking water. This amount can be present in a nutritional complete feed as such, at a level of 0.01 to 5 kg per ton of that feed material, or may, for example, be present in a concentrated feed material (exceeding 5 kg/ton feed material) as long as the amount per total daily intake of feed is between 0.01 and 5 kg ABM mycelium per ton. In particular, the ABM mycelium is fed at an amount of 0.05 to 2 kg per ton of total daily intake of feed. These amounts appear to suffice for use according to the current disclosure.

In a further embodiment, the one or more agent-affecting bacteria belonging to enterobacteriaceae are present in present in the animal feed in an amount of at least 0.01 kg per ton of total daily intake, preferably at least 0.02, more preferably at least 0.05 kg per ton, and at most 10 kg per ton of total daily intake of feed, preferably at most 5 kg per ton, more preferably at most 2 kg per ton, and most preferably at most 1 kg per ton of total daily intake.

The disclosure further pertains to the use of the composition of the disclosure against resistant bacteria of the Enterobacteriaceae, in particular, of Salmonella and/or Escherichia. With the term “resistant bacteria” is meant bacteria that are resistant to conventional antibiotics. Examples of such resistant bacteria include cefotaxime-resistant Escherichia coli, carbapenem-resistant enterobacteriaceae and extended spectrum beta lactamase-producing Escherichia coli (ESBL-producing E. coli). Preferably, the disclosure pertains to the use of the inventive composition in animal feed against resistant bacteria of the Enterobacteriaceae, in particular, of Salmonella and/or Escherichia.

### Examples

Example 1 describes an in vitro model study for assessing the effect of an antimicrobial on bacterial growth.

Example 2 describes an in vivo study for assessing the effect of ABM mycelium combined with organic acids on bacterial shedding.

Example 3 describes a second in vivo study for assessing the effect of ABM mycelium combined with organic acids on bacterial shedding.

Example 4 describes an in vitro study on bacterial growth with different treatments.

Example 5 describes an in vivo study for assessing the effect of ABM mycelium combined with organic acids on bacterial shedding.

Example 6 describes an in vivo study with broilers assessing the transmission of Salmonella.

### BRIEF DESCRIPTION OF THE DRAWINGS

FIG. 1 shows the effect of ABM mycelium combined with organic acids on the shedding of Salmonella.

FIG. 2 shows the effect of ABM mycelium combined with organic acids on diarrhea.

FIG. 3 shows the effect of ABM mycelium combined with organic acids on the feed intake.

FIG. 4 shows the effect of ABM mycelium combined with organic acids on the feed efficacy.

FIG. 5 shows the effect of ABM mycelium combined with organic acids on the shedding of enterobacteriaceae in further in vivo studies.

### DETAILED DESCRIPTION

### Example 1

Example 1 describes an in vitro model study for assessing the effect of ABM mycelium on bacterial adhesion. In this method the adhesion of Salmonella typhimurium to ABM mycelium is assessed.

Use was made of a 96-well plate on which the ABM mycelium was coated. For this, the ABM mycelium (in this and each case below a fermented rye product was actually used, in which product the amount of ABM mycelium was about 15% w/w) was suspended in PBS to a final concentration of 1% (w/v) and mixed thoroughly. Subsequently the suspension was centrifuged to remove insoluble material. Thereafter, the supernatant was used for coating the wells of the microtiter plate. For the adhesion assessment, a Salmonella typhimurium suspension was added to the microtiter plate. The plate was then incubated for 30 minutes and after this incubation step washed with PBS. Subsequently growth medium was added to the wells and the time to onset OD600 value was determined. The optical density (OD) measurement was used as a tool to compare numbers of adhered bacteria to the coated wells of the 96-well plate with different compounds. The initial cell density of adhered bacteria correlates with the time-dependent detection of the growth by optical density measurement. A shorter time to onset OD600 value represents more adhesion of bacteria to the substrate, and hence an expected higher decrease of in vivo growth.

The results for the test with Salmonella typhimurium showed that the average time to onset OD600 was 4.9 hours (±0.3 hour) as compared to the control (only PBS), which had an average time to onset OD600 of 7.3 hours (±0.1 hour). About twenty other compounds that were suspected of having a potential effect an adhesion (compounds not indicated in this example) showed an average time to onset OD600 generally between 5 and 8.5 hours.

In a second in vitro study, the test was repeated, and additionally the effect on Salmonella enteritidis and E. coli was measured. Also, the amount of ABM mycelium was used in the full amount (see above; denoted “100%”), half of this amount (“50%”) and a quarter of this amount (“25%”). The results are indicated here beneath in Table 1.

TABLE 1
Effect of ABM mycelium in various amounts on
the adhesion of various enterobacteriaceae,
by measuring the time to onset OD600 in hours.

| Compound | S. typhimurium | S. enteritidis | E. coli |
|---|---|---|---|
| Control | 7.0 | 6.3 | 7.1 |
| ABM 100% | 6.0 | 5.5 | 5.7 |
| ABM 50% | 5.7 | 5.5 | 5.9 |
| ABM 25% | 5.7 | 5.5 | 6.4 |

From the model studies it appears that mycelium of ABM has a significant effect on the adhesion of various enterobacteriaceae. The effect appears to be independent of the type of bacterium despite the fact that, in particular, the Escherichia bacteria are of a completely different species than the Salmonella bacteria. The amount of ABM mycelium does not appear to be critical to obtain the adhesion effect as such.

### Example 2

Example 2 describes an in vivo study for assessing the effect of ABM mycelium combined with organic acids on bacterial shedding. In this study it was assessed whether the effect seen in vitro (see Example 1) indeed corresponds to in vivo bacterial shedding. In particular, it was assessed whether by introducing ABM mycelium in the feed of the pigs, in this case combined with an organic acids blend, the shedding of viable bacteria could be reduced. As controls, a negative control using the regular feed was used, and as a positive control the same feed with added butyrate, a particular short chain fatty acid that is commercially used in poultry feed to reduce bacterial shedding. The organic acid blend was a regular C₁-C₁₆ organic acid blend containing a combination of formic and lactic acid, added at 4 liters per 100 kg.

A total of 24 Topi*Hypor boar piglets were used. Only healthy male animals that did not receive antibiotics and that were negative for Salmonella (determined by qualitative examination of the feces) were included in the study. Animals were identified by uniquely numbered ear tags. Animals were divided over three treatment groups (8 animals per group) by weight and litter.

Piglets were individually housed (0.8×1.6 m) directly after weaning (24 days of age+/−3 days) in pens containing tenderfoot slatted floors. The first 24 hours after weaning continuous light was provided, thereafter 16 hours light and 8 hours darkness. Piglets received feed and drinking water ad lib. The different treatments were administered in the feed during the total study period (from weaning until the end of the study) as indicated below in Table 2.

TABLE 2
Feed treatments

| Treatment | No. of animals | Additives | Inclusion level |
|---|---|---|---|
| Negative control | 8 | — | — |
| Butyrate | 8 | Butyrate + | 6 kg/ton + |
| | | acid blend | 4 L/ton |
| ABM mycelium | 8 | ABM + | 2 kg/ton + |
| | | acid blend | 4 L/ton |

After 10 days piglets were orally infected with Salmonella typhimurium (in BHI medium) given by a pre-inoculated feed matrix containing 1 ml 1*10⁹ cfu/ml. Oral infection was performed in this way during 7 consecutive days.

Fecal sampling was performed at day 1, 2, 3, 4, and 7 post Salmonella infection. Samples were stored at 4 degrees and analyzed the next day. Samples were diluted and homogenized in BPW containing novobiocin. Serial dilutions were made and plated onto selective chromogenic agar plates, and incubated o/n at 37° C. Typical Salmonella colonies were counted and the amount (cfu/gram) was calculated. Of each sample two presumptive Salmonella colonies were confirmed by qPCR for both Salmonella and Salmonella typhimurium. When no colonies were observed in the lowest dilution plates the samples were screened for Salmonella presence (qualitative) after pre-enrichment by the conventional MSRV/XLD method.

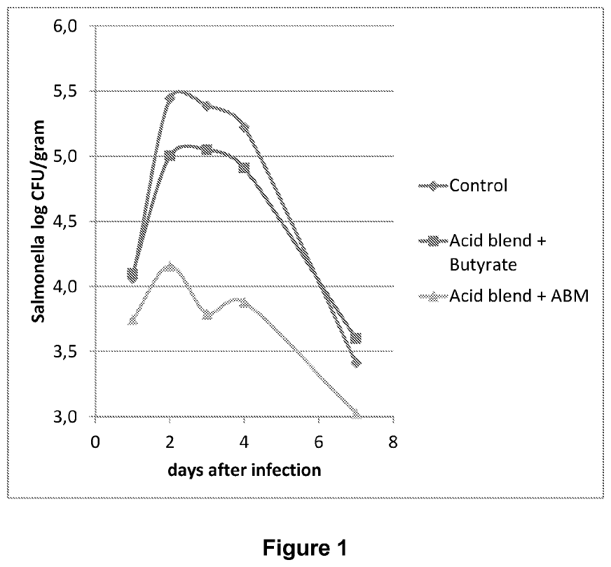

The results are indicated in FIG. 1, which shows the effect of ABM mycelium combined with organic acids on the shedding of Salmonella. It appears that the combination of mycelium of ABM and organic acids indeed has a significant effect on the shedding of viable salmonella bacteria. In particular, the effect is very large when compared to butyrate, a compound that is used in poultry for this purpose. It is thus also clear that the in vitro model (Example 1) is predictive for the in vivo reduction of bacterial shedding.

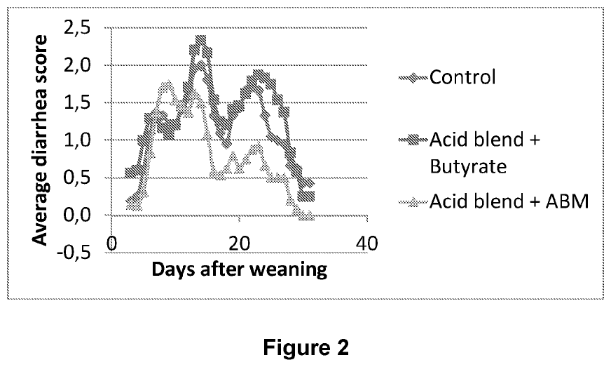

FIG. 2 shows the effect on diarrhea. A feces scoring was performed daily from day 3 after weaning until the end of the study. Diarrhea score was determined as: 0=normal feces; 1=flat feces; 2=wet feces; 3=watery feces. The results as depicted in FIG. 2 show a significant reduction of the ABM mycelium on diarrhea.

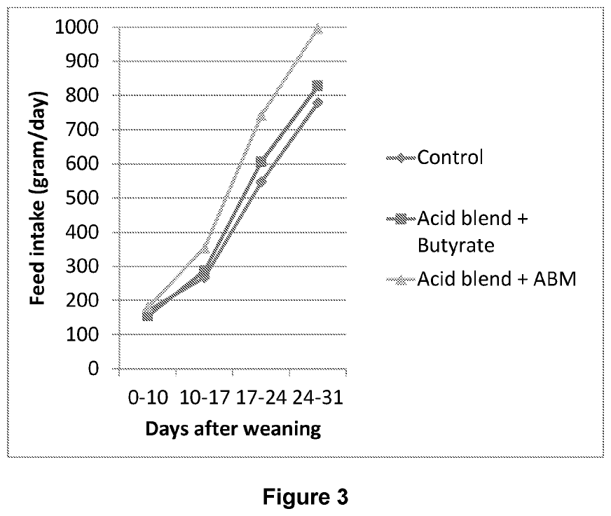

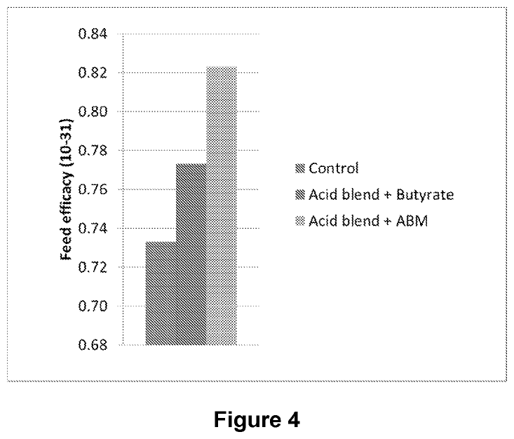

To assess performance, piglets were inspected daily. Body weight and feed intake were determined at weaning, before infection, and 7, 14, and 21 days after infection (day 0, 10, 17, 24, and 31). Feed efficacy was determined as gram growth/gram feed intake. FIG. 3 shows the effect of ABM mycelium combined with organic acids on the feed intake. FIG. 4 shows the effect of ABM mycelium combined with organic acids on the feed efficacy. The results show a significant positive impact on performance due to the presence of ABM mycelium in the feed.

### Example 3

Example 3 describes a second in vivo study for assessing the effect of ABM mycelium combined with organic acids on bacterial shedding. In this study, as a positive control the acid blend on itself was used (thus without the ABM mycelium).

A total of 36 Topi*Hypor boar piglets were used. Only healthy male animals that did not receive antibiotics and that were negative for Salmonella (determined by qualitative examination of the feces) were included in the study. Animals were identified by uniquely numbered ear tags. Animals were divided over three groups (12 animals per group) by weight and litter.

Piglets were individually housed (0.8×0.8 m) directly after weaning (24 days of age+/−3 days) in pens containing tenderfoot slatted floors. The first 24 hours after weaning continuous light was provided, thereafter 16 hours light and 8 hours darkness. Piglets received feed and drinking water ad lib. The different treatments were administered in the feed during the total study period (from weaning until the end of the study) as indicated below in Table 3.

TABLE 3
Feed treatments

| Treatment | No. of animals | Acid blend | ABM |
|---|---|---|---|
| Negative control | 12 | None | — |
| Acid blend | 12 | 4 L/ton | — |
| Acid blend + ABM mycelium | 12 | 4 L/ton | 2 kg/ton |

After 8 days piglets were orally infected with Salmonella typhimurium (in BHI medium) given by a pre-inoculated feed matrix containing 1 ml 1*10⁹ cfu/ml. Oral infection was performed in this way during 7 consecutive days.

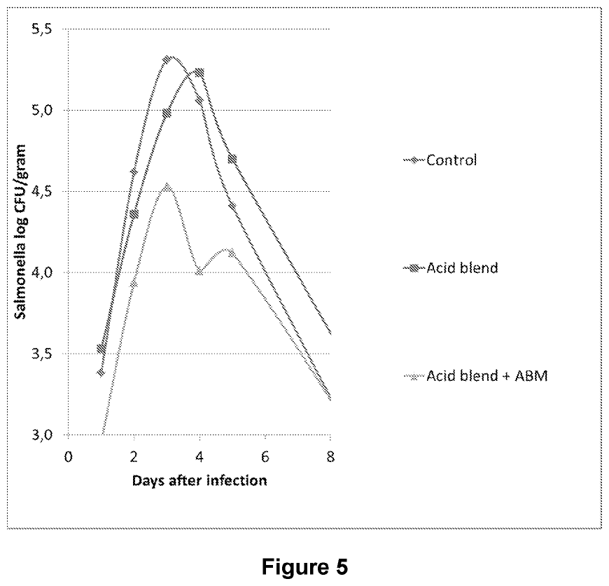

Fecal sampling was performed at day 1, 2, 3, 4, and 5 post Salmonella infection. Samples were stored at 4 degrees and analyzed the next day. Samples were diluted and homogenized in BPW containing novobiocin. Serial dilutions were made and plated onto selective chromogenic agar plates, and incubated o/n at 37° C. Typical Salmonella colonies were counted and the amount (cfu/gram) was calculated. Of each sample two presumptive Salmonella colonies were confirmed by qPCR for both Salmonella and Salmonella typhimurium. When no colonies were observed in the lowest dilution plates the samples were screened for Salmonella presence (qualitative) after pre-enrichment by the conventional MSRV/XLD method. The results are indicated in FIG. 5 and correspond to the results as indicated in FIG. 1.

The above in vivo experiment was repeated to assess the effect on Escherichia coli shedding by pigs. The experiment was run in correspondence with the salmonella experiment as described here above, with 10 animals being used per group. The results showed that on the day of artificial E. coli infection, none of the animals were positive in their feces for E. coli. At day 12, over 70% of the animals were positive in each group. Two days later, in the two control groups (negative control and acid blend group) the percentage of positive animals was 60%, whereas in the ABM group no shedders (0% of the animals tested positive for E. coli) were present at all.

### Example 4

An in vitro model study was conducted according to the protocol described in Example 1. In this method the adhesion of Salmonella typhimurium and of Escherichia coli to ABM mycelium on rye, to β-1,4-mannobiose, and to three combinations of ABM mycelium on rye and β-1,4-mannobiose with different combinations, i.e., 75:25, 50:50 and 25:75. The total amount of the agents is the same in all experiments.

The results for the test with Salmonella typhimurium are shown in the Table below.

TABLE 4
Effect of ABM mycelium on rye and β-1,4-mannobiose
in various amounts on the adhesion of various enterobacteriaceae,
by measuring the time to onset OD600 in hours.

| Compound | S. typhimurium | E. coli |
|---|---|---|
| Control | 6.95 | 7.10 |
| ABM 100%: β-1,4-mannobiose 0% | 5.95 | 5.70 |
| ABM 0%: β-1,4-mannobiose 100% | 5.90 | 7.30 |
| ABM 25%: β-1,4-mannobiose 75% | 5.70 | 5.95 |
| ABM 50%: β-1,4-mannobiose 50% | 5.75 | 5.80 |
| ABM 25%: β-1,4-mannobiose 75% | 5.75 | 6.35 |

The model studies show that mycelium of ABM on rye and β-1,4-mannobiose have a significant effect on the adhesion of Salmonella typhimurium. The onset OD600 measured for the different combinations of ABM mycelium on rye and β-1,4-mannobiose is significantly lower than the onset OD600 measured for ABM mycelium and β-1,4-mannobiose alone.

The experiments with E. coli reveal that ABM mycelium on rye considerably reduces the onset OD600 compared to the control. The experiments also show that β-1,4-mannobiose has an onset OD600 exceeding the onset observed for the control, revealing that β-1,4-mannobiose does not have an effect on the adhesion of E. coli. The combinations of ABM mycelium on rye and β-1,4-mannobiose show a considerably lower onset OD600 than the control and the β-1,4-mannobiose alone. It is further noted that the onset OD600 values of the combinations is lower than what is expected based on a linear relationship between the onset OD values of ABM mycelium on rye alone and β-1,4-mannobiose alone.

### Example 5

An in vivo study was conducted according to the protocol described in Example 2, except that 12 animals per treatment group were used and the piglets were selected based on the presence of cefotaxime-resistant Escherichia coli; the selected animals were infected with Salmonella enteritidis after 5 days. In this study the shedding of cefotaxime-resistant Escherichia coli to ABM mycelium on rye and to a combination of ABM mycelium on rye and β-1,4-mannobiose at 50:50. The organic acid blend was a regular C₁-C₁₆ organic acid blend containing a combination of formic and lactic acid. The total amount of the agents is the same in all experiments.

The results for the test with cefotaxime-resistant Escherichia coli are shown in the Table below.

TABLE 5
Effect of ABM mycelium on rye and β-1,4-mannobiose
on the shedding of cefotaxime-resistant Escherichia
Coli, by measuring over the first 4 and over 16 days

| Compound | Amount (kg/ton feed) | 1-4 days | 16 days |
|---|---|---|---|
| Control | — | 2.0 | 2.1 |
| Acid blend | 4 | 1.4 | 1.5 |
| ABM 100% | 2 | 0.8 | 1.0 |
| ABM 50%: | 2 | 0.5 | 0.9 |
| β-1,4-mannobiose 50% | | | |

The study shows that mycelium of ABM on rye with or without β-1,4-mannobiose have a significant effect on the shedding of cefotaxime-resistant Escherichia coli. The acid blend alone does not provide a significant reduction of the shedding of cefotaxime-resistant Escherichia coli.

### Example 6

An in vivo study was conducted using two groups, each group comprising 6 replicating pens with 30 birds. Three birds in each pen were infected with Salmonella enteritidis (seeder birds). The broilers were fed with a conventional broiler diet during 42 days. One group of broilers was treated with ABM mycelium on rye and an organic acid blend. The organic acid blend was a regular C₁-C₁₆ organic acid blend containing a combination of formic and lactic acid. The transmission of Salmonella to non-seeder birds was established by determining the number of infected or positive birds after 28 and 42 days.

After 28 days, the control (untreated) group consisted of 83% of infected birds, whereas the treated group contained 55% of infected birds. After 42 days, 60% of the birds were infected in the control group and 35% of positive birds in the treated group. This clearly demonstrates that the treatment aids in the containment of the Salmonella in the broilers.

1.-9. (canceled)

10. A composition comprising:
mycelium of Agaricus Blazei Murill (“ABM mycelium”); and
an agent-affecting bacteria belonging to Enterobacteriaceae.

11. The composition of claim 10, wherein the ABM mycelium is grown on grain.

12. The composition of claim 10, wherein the agent-affecting bacteria belonging to Enterobacteriaceae is selected from the group consisting of formic acid, lactic acid, benzoic acid, and β-1,4-mannobiose.

13. The composition of claim 11, wherein the agent-affecting bacteria belonging to Enterobacteriaceae is selected from the group consisting of formic acid, lactic acid, benzoic acid, and β-1,4-mannobiose.

14. The composition of claim 10, having a weight ratio of the ABM mycelium to the agent-affecting bacteria belonging to Enterobacteriaceae in the compositions from at least 0.01 to at most 50.

15. The composition of claim 11, having a weight ratio of the ABM mycelium to the agent-affecting bacteria belonging to Enterobacteriaceae in the compositions from at least 0.01 to at most 50.

16. The composition of claim 12, having a weight ratio of the ABM mycelium to the agent-affecting bacteria belonging to Enterobacteriaceae in the compositions from at least 0.01 to at most 50.

17. The composition of claim 13, having a weight ratio of the ABM mycelium to the agent-affecting bacteria belonging to Enterobacteriaceae in the compositions from at least 0.01 to at most 50.

18. The composition of claim 10, wherein the composition is an animal feed for an animal.

19. The composition of claim 18, wherein the ABM mycelium is present therein in an amount between 0.01 and 5 kg per metric ton of total daily intake for the animal.

20. A premix of the composition of claim 18.

21. The composition of claim 10, wherein the composition is a feed additive.

22. The composition of claim 12, wherein the agent-affecting bacteria belonging to Enterobacteriaceae is β-1,4-mannobiose.

23. The composition of claim 13, wherein the agent-affecting bacteria belonging to Enterobacteriaceae is β-1,4-mannobiose.

24. The composition of claim 14, wherein the agent-affecting bacteria belonging to Enterobacteriaceae is β-1,4-mannobiose.

25. The composition of claim 15, wherein the agent-affecting bacteria belonging to Enterobacteriaceae is β-1,4-mannobiose.

26. The composition of claim 16, wherein the agent-affecting bacteria belonging to Enterobacteriaceae is β-1,4-mannobiose.

27. The composition of claim 17, wherein the agent-affecting bacteria belonging to Enterobacteriaceae is β-1,4-mannobiose.

28. A kit-of-parts comprising:
a first constituent part comprising Agaricus Blazei Murill (ABM) mycelium;
a second constituent part comprising at least one agent(s)—affecting bacteria belonging to Enterobacteriaceae; and, optionally,
an instruction to orally administer both the constituent parts to an animal.
